(19) United States
(12) Patent Application Publication
(10) Pub. No.: US 2023/0345991 A1
(43) Pub. Date: Nov. 2, 2023

(54) METHOD OF MAKING FUNCTIONAL FIBER HAVING IMPROVED DEWATERING EFFICIENCY

(71) Applicant: CORN PRODUCTS DEVELOPMENT, INC, WESTCHESTER, IL (US)

(72) Inventors: William BORCHERT, Bridgewater, NJ (US); Matthew SILLICK, Bridgewater, NJ (US)

(21) Appl. No.: 18/016,693

(22) PCT Filed: Jul. 29, 2021
(86) PCT No.: PCT/US2021/043602

§ 371 Date: Jan. 18, 2023

Related U.S. Application Data
(60) Provisional application No. 63/060,809, filed on Aug. 4, 2020.

(51) Int. Cl. A61K 8/02; A23L 33/22; A61Q 19/00; A61K 8/9789; C11D 3/382

(52) U.S. Cl. CPC A23L 33/22; A61K 8/027; A61K 8/9789; A61Q 19/00; C11D 3/382; A61K 2800/10

(57) ABSTRACT

The technology disclosed in this specification pertains to methods for increasing the dewatering efficiency of a sheared plant fiber, for example citrus fiber, by shearing the plant material in a slurry of water and organic solvent. The plant fibers obtained from the method provide significant viscosity even when using little alcohol. The obtained citrus fibers may be used in edible, cosmetic, household, or an industrial compositions.

The technology disclosed in this specification pertains to functionalized plant fiber and methods of making the same, and more particularly to functionalized citrus fibers.

Plant fibers can be processed so that it changes the rheology and sensory properties of food products, sometimes called functionalization. One process for functionalizing a fiber involves applying shear to a fiber slurry. This process increases the water holding capacity of fiber in the slurry and makes it difficult to dewater the fiber.

The technology disclosed in this specification provides methods for increasing the dewatering efficiency of a sheared fiber.

In any embodiment described in this specification a method for increasing the dewatering efficiency of a sheared plant fiber comprises (i) dispersing a comminuted depectinated plant fiber in a solution comprising an organic solvent to form a slurry having a solid phase and an aqueous phase; (ii) applying shear to the slurry; and (iii) recovering the plant fiber from the slurry. Suitable organic solvents for use in the methods disclosed in this specification include, but are not limited to, low molecular weight solvents with H-bonding capability and low flash point. Suitable organic solvents for use in the methods disclosed in this specification include, but are not limited to, short chain alcohols such as ethanol, butanol, propanol, methanol and mixtures thereof. In any embodiment of the method disclosed in this specification the organic solvent is isopropanol. In any embodiment a depectinated plant material useful as a solid phase within the slurries described in this specification has a moisture content of from about 0.25 to about 15% or about 0.5% to about 10% or from about 1% to about 8% or from about 1% to about 5% (d.b.) citrus fiber.

In any embodiment, the methods described in this specification comprise a slurry liquid phase having a solid phase and a liquid phase comprising water and an organic solvent. In any embodiment described in this specification the method comprises a slurry having a liquid phase comprising from about 5% to about 50% organic solvent, or from about 5% to about 25%, or from about 5% to about 15% or from about 5% to about 12.5% or from 5% to about 10% or from about 5% to about 7.5%.

In any embodiment, the methods described in this specification obtain a depectinated plant fiber from an unprocessed plant fiber having at least some of the pectin. In any embodiment described in this specification, a depectinated fiber retains at least some of the pectin present in an unprocessed plant fiber. In any embodiment of the methods described in this specification a depectinated plant fiber is a depectinated citrus fiber, obtained from, for example, but not limited to lemon, orange or lime or mixtures thereof. In any embodiment described in this specification, plant fibers may be obtained from parenchymal cells of a plant, or from skin or rind of a plant or from the peel a citrus fruit. In any embodiment of the method, as described in this specification, a depectinated citrus fiber has a pectin content of less than about 19% by weight of the fiber (d.b.), or less than about 15% or less than about 10% or less than about 5% or from about 1% to about 19% or from about 1% to about 15% or from less than about 1% to about 10% or from about 1% to about 5%, or from about 1% or about 2% or about 3% or about 4%. Depectinated plant fibers are comminuted using any standard process known in the industry including grinding. The comminuted depectinated plant fibers may be comminuted before or after depectination.

In any embodiment pectin is removed from plant fiber material using a process to solubilize pectin so that it can be washed out of the plant fiber material. Any suitable process for rendering pectin soluble may be used including washing the plant fiber material in an acidic solution. In any embodiment described in this specification a plant fiber material may be depectinated using a method comprising washing a plant fiber material in a solution having pH below about 3, or below about 2 or from about 1 to about 2 or from 1.5 to about 2 or about 1.8. In any embodiment described in this specification a plant fiber material is depectinated by washing a pectin containing plant material at a temperature above about 50° C. or above about 60° C. or from about 60° C. to about 80° C. or from about 65° C. to about 75° C. Depectinated plant fiber may be washed to further bleach the material, which may also remove soluble components (e.g. hemicellulose) and insoluble components (e.g. lignin or oil) from the plant fiber material. In any embodiment described in this specification, a depectinated citrus peel may be washed prior to applying the shear in a solution comprising from about 5% to about 30% v/v, or from about 5% to about 25% or from about 5% to about 20% or from about 10% to about 20% or from about 12% to about 18% or about 15% of an organic acid, or a beaching agent, including for example, but not limited to, peracetic acid or hydrogen peroxide.

In any embodiment the methods described in this specification comprise applying a shear energy via a pressure drop across a restricted orifice or nozzle and the pressure drop is at least about 100 to about 5000 bar, or from about 100 to about 2500, or about 100 to about 1000, or about 100 to about 750 or about 100 to about 500 or about 300 to about 1000 bar. In any embodiment the described methods apply shear using any suitable mixing or homogenizing device. In any embodiment the methods described in this specification applying a shear to a slurry using a rotor/stator homogenizer rotating at from about 1,000 to about 20,000 rpm, or from about 1,000 to about 15,000, or from about 1,000 to about 10,000, or from about 2,500 to about 10,000, or from about 5,000 to about 10,000. In any embodiment described in this specification, a slurry is sheared in an apparatus being, for example, but not limited to mixer or homogenizer for at least about 1 minute, or at least about 5 minutes or at least about 10 minutes or at least about 15 minutes or at least about 30 minutes, or from about 5 to 45 minutes, or from about 10 to about 30 minutes. In any method disclosed in this specification a slurry is sheared, for example including but not limited to, in a homogenizer in one pass, at least one pass, or at least two passes.

In any embodiment described in this specification a sheared plant fiber slurry is dewatered using any means suitable for separating a solid phase from a liquid phase, including but not limited to sieves and mechanical means such as presses, and centrifuges. In any embodiment of the described methods a dewater step removes at least about 10% of the liquid from the dispersed fiber or at least about 15% or at least about 20% or at least about 25% or at least about 30% or at least about 40%, or from about 10% to about 40%, or from about 15% to about 40% of from about 20% to about 40%.

In any embodiment, the methods described in this specification comprise drying the recovered plant fiber. In any embodiment described in this specification the plant fiber has moisture content of less than about 10% or from about 1% to about 10% or from about 4% to about 10%, or from about 5% to about 9% or from about 6% to about 8%.

The technology described in this specification also pertains to functionalized plant fiber material, or functionalized citrus fiber material, or functionalized orange fiber, or lemon fiber, or lime fiber, or mixtures thereof. In any embodiment a functionalized plant fiber as described in this specification may be made by any method described in this specification. In any embodiment a plant fiber is a citrus fiber and the methods described above obtain a recovered citrus fiber having a median particle size of less than about 45 microns, or less than about 43 microns, or from about 35 to about 45 microns, or from about 35 to about 43 microns, or from about 38 to about 43 microns.

In any embodiment a plant fiber is a citrus fiber and the methods described above obtain a recovered citrus fiber capable of forming an aqueous dispersion at 1% citrus fiber (d.b.) having a 90th percentile particle size of from less than about 100 microns, or less than about 98 microns, or from about 80 to about 100 microns, or from about 85 to about 100 microns, or from about 90 to about 100 microns, or from about 95 to about 100 microns, or from about 95 to about 98 microns.

In any embodiment a plant fiber is a citrus fibers and the methods described above obtain a recovered citrus fiber capable of forming an aqueous dispersion at 1% citrus fiber (d.b.) having a viscosity at 10 s⁻¹ of greater than about 3 Pa.s, or greater than about 3.1 Pa.s, or from about 3.0 to about 3.5, or from about 3.1 to about 3.2 Pa.s.

In any embodiment, a plant fiber is a citrus fiber and the methods described above obtain a recovered citrus fiber capable of forming an aqueous dispersion at 1% citrus fiber (d.b.) having a Bostwick distance of less than about of less than about 8.0 or less than about 6.0 cm, or less than about 5.0, or from about 4.0. to about 8.0, or from about 4.0 to about 6.0, or from about 4.0 to about 5.0.

In any embodiment, a plant fiber is a citrus fiber and the methods described above obtain a recovered citrus fiber capable of forming an aqueous dispersion at 1% citrus fiber (d.b.) having an elastic modulus (G′) at 1 rad/s of greater than about 200 Pa or greater than about 250 Pa, or greater than about 275 Pa, or from about 200 to about 310 Pa or from about 250 to about 310 Pa, or from about 275 to about 310 Pa, or from about 280 to about 300 or from about 280 to about 295, or from about 285 to about 290 Pa;

In any embodiment, a plant fiber is a citrus fiber and the methods described above obtain a recovered citrus fiber having a water holding capacity of from about 60 to about 100 (g/g) or from about 70 to about 100 or from about 80 to about 100, or from about 85 to about 95.

In any embodiment, the citrus fibers described in this specification and made by the processes described in this specification are useful in edible, cosmetic, household (e.g. a cleanser), and industrial applications. In any embodiment, the citrus fibers, described in this specification and made by the processed described in this specification are useful to provide viscosity to liquid, or to bind water, or to stabilize a suspension of solids in a liquid phase.

In any embodiment a composition comprising a plant fiber as described in any of the foregoing claims and a second edible ingredient, wherein optionally the composition is selected from the group consisting of beverages, sauces, dressings, soft-baked and cold-pressed bars, beverages, instant mixes, processed and packaged meat products and meat analog products, ice creams, frozen desserts, and baked goods including gluten free goods.

In any embodiment a composition may comprise, as a second edible ingredient any ingredient commonly used in a gluten free baked good including, but not limited to, oil, water, sweeteners, eggs (whole, or egg whites or egg yolks, whether in natural, powdered, or other form), leavening agents (yeasts and chemical leavening agents), salts, flavoring agents, preservatives, and fibers. Illustrative, non-limiting ingredients include, oils such as canola oil, corn oil, or vegetable oil. Illustrative, non-limiting ingredients include, sweeteners in solid or liquid form including but not limited to sucrose, or corn syrup or high fructose corn syrup and include steviol glycosides, fructose isomers (e.g. allulose, tagatose), high potency sweeteners such as erythritol, and other low caloric or non-caloric sweeteners. Illustrative, non-limiting ingredients include starches from any common source including corn, tapioca, rice, potato, sago and from pulses (including high and low amylose variants). Illustrative, non-limiting ingredients include fruit preparations such as pureed, thickened fruit preparations or fruit juices. Illustrative, non-limiting ingredients include gums and other hydrocolloids. Such ingredients may be used in suitable amounts in amount from about 0.1 to about 99% and all ranges in between.

The technology disclosed in this specification is further described in the following aspects, which are intended to be illustrative, and are not intended to limit the full scope of the claims and their equivalents.

In a first aspect, the technology disclosed in this specification pertains to a method for increasing the dewatering efficiency of a sheared plant fiber comprising: (i) forming a slurry having a solid phase and a liquid phase comprising a comminuted depectinated plant material, an aqueous solution, and an organic solvent; (ii) applying shear to the slurry; and (iii) recovering the plant fiber from the slurry; optionally wherein the plant fiber is obtained from a plant material is selected from the group consisting of citrus peel, or is orange peel, lemon peel, lime peel, and mixtures thereof.

In a second aspect, the technology disclosed in this specification pertains to the method of the first aspect wherein the solvent is an alcohol or optionally isopropyl alcohol, methanol, ethanol, propanol, butanol, and mixtures thereof.

In a third aspect, the technology disclosed in this specification pertains to the method of first or second aspect wherein the depectinated plant fiber has a pectin content of less than about 19% by weight (d.b.), or less than about 15% or less than about 10% or less than about 5% or from about 1% to about 19% or from about 1% to about 15% or from less than about 1% to about 10% or from about 1% to about 5%, or from about 1% or about 2% or about 3% or about 4%.

In a fourth aspect, the technology disclosed in this specification pertains to the method any one of the first to third aspects wherein the slurry’s liquid phase comprising water and the organic solvent, optionally wherein the slurry comprises from about 5% to about 50% organic solvent, or from about 5% to about 25%, or from about 5% to about 15% of from about 5% to about 12.5% or from 5% to about 10% or from about 5% to about 7.5%.

In a fifth aspect, the technology disclosed in this specification pertains to the method of any one of the first to fourth aspects wherein the shear energy is applied via a pressure drop across a restricted orifice or nozzle and the pressure drop is at least about 100 to about 5000 bar, or from about 100 to about 2500, or about 100 to about 1000, or about 100 to about 750 or about 100 to about 500 or about 300 to about 1000 bar.

In a sixth aspect, the technology disclosed in this specification pertains to the method of any one of the first to fifth aspects wherein the shear energy is applied to the slurry by a rotor/stator homogenizer rotating at from about 1,000 to about 20,000 rpm, or from about 1,000the shear applied to the slurry is from about 1,000 to about 20,000 rpm, or from about 1,000 1o about 15,000, or from about 1,000 to about 10,000, or from about 2,500 to about 10,000, or from about 5,000 to about 10,000, and wherein optional the slurry is shear for at least about 1 minute, or at least about 5 minutes or at least about 10 minutes or at least about 15 minutes or at least about 30 minutes, or from about 5 to 45 minutes, or from about 10 to about 30 minutes.

In a seventh aspect, the technology disclosed in this specification pertains to the method of any one of the first to sixth aspects wherein the slurry is sheared in one pass, at least one pass, or at least 2 passes.

In an eighth aspect, the technology disclosed in this specification pertains to the method of any one of the first to seventh aspects wherein the slurry’s solid phase is made of from about 0.25 to about 15% comminuted depectinated plant material or about 0.5% to about 10% or from about 1% to about 8% or from about 1% to about 5% (d.b.).

In a ninth aspect, the technology disclosed in this specification pertains to the method of any one of the first to eighth aspects wherein recovering the plant fiber removes at least about 10% of the liquid from the dispersed fiber or at least about 15% or at least about 20% or at least about 25% or at least about 30% or at least about 40%, or from about 10% to about 40%, or from about 15% to about 40% of from about 20% to about 40%.

In a tenth aspect, the technology disclosed in this specification pertains to the method of any one of the first to ninth aspects further comprising drying the recovered plant fiber, optionally wherein the dried plant fiber has moisture content of less than about 10% or from about 1% to about 10% or from about 4% to about 10%, or from about 5% to about 9% or from about 6% to about 8%.

In an eleventh aspect, the technology disclosed in this specification pertains to the method of any one of the first to tenth aspects further comprising treating a plant material or a dried filer to solubilize pectin and filtering the plant material to remove pectin.

In a twelfth aspect, the technology disclosed in this specification pertains to the method of any one of the first to eleventh aspects further comprising removing pectin from a plant material to obtain the depectinated plant material by solubilizing pectin in the peel in an acidic solution, having pH below about 3, or below about 2 or from about 1 to about 2 or from 1.5 to about 2 or about 1.8.

In a thirteenth aspect, the technology disclosed in this specification pertains to the method of any one of the first to twelfth aspects further comprising removing pectin from a plant material to obtain the depectinated plant material by solubilizing pectin within the peel in an acidic solution at temperature above about 50° C. or above about 60° C. or from about 60° C. to about 80° C. or from about 65° C. to about 75° C.

In a fourteenth aspect, the technology disclosed in this specification pertains to the method of any one of the first to thirteenth aspects further comprising washing a depectinated plant material prior to applying the shear plant material, optionally wherein the washing step whitens the plant material.

In a fifteenth aspect, the technology disclosed in this specification pertains to the method of any one of the first to fourteenth aspects further comprising washing a depectinated plant material prior to applying the shear in a solution comprising from about 5% to about 30% v/v, or from about 5% to about 25% or from about 5% to about 20% or from about 10% to about 20% or from about 12% to about 18% or about 15% or an organic acid.

In a sixteenth aspect, the technology disclosed in this specification pertains to the method of any one of the first to fifteenth aspects further comprising washing a depectinated plant material prior to applying the shear in a solution containing and a bleaching agent, or peracetic acid or hydrogen peroxide.

In a seventeenth aspect, the technology disclosed in this specification pertains to the method of any one of the first to sixteenth aspects wherein the process obtains a recovered plant fiber or citrus fiber capable of forming an aqueous dispersion at 1% recovered fiber (d.b.) having a median particle size of less than about 45 microns, or less than about 43 microns, or from about 35 to about 45 microns, or from about 35 to about 43 microns, or from about 38 to about 43 microns.

In an eighteenth aspect, the technology disclosed in this specification pertains to the method of any one of the first to seventeenth aspects wherein the process obtains a recovered plant fiber or citrus fiber capable of forming an aqueous dispersion at 1% recovered fiber (d.b.) having a 90th Percentile particle size of from less than about 100 microns, or less than about 98 microns, or from about 80 to about 100 microns, or from about 85 to about 100 microns, or from about 90 to about 100 microns, or from about 95 to about 100 microns, or from about 95 to about 98 microns.

In a nineteenth aspect, the technology disclosed in this specification pertains to any one of the first to eighteenth aspects wherein the process obtains a recovered plant fiber or citrus fiber, capable of forming an aqueous dispersion at 1% recovered fiber (d.b.) having a viscosity at 10 s⁻¹ of greater than about 3 Pa.s, or greater than about 3.1 Pa.s, or from about 3.0 to about 3.5, or from about 3.1 to about 3.2 Pa.s.

In a twentieth aspect, the technology disclosed in this specification pertains to the method of any one of the first to nineteenth aspects wherein the process obtains a recovered plant fiber or citrus fiber capable of forming an aqueous dispersion at 1% recovered fiber (d.b.) having a Bostwick distance of Bostwick distance of less than about 8 or less than about 6.0 cm, or less than about 5, or from about 4. to about 8.0, or from about 4.0 to about 6.0, or from about 4.0 to about 5.0.

In a twenty-first aspect, the technology disclosed in this specification pertains to the method of any one of the first to twentieth aspects wherein the process obtains a recovered plant fiber or citrus fiber having a water holding capacity of from about 60 to about 100 g/g, or from about 70 to about 100 or from about 80 to about 100, or from about 85 to about 95.

In a twenty-second aspect, the technology disclosed in this specification pertains to the method of any one of the first to twenty-first aspects wherein the process obtains a recovered plant fiber or citrus fiber capable of forming an aqueous dispersion at 1% recovered fiber (d.b.) having an elastic modulus (G′) at 1 rad/s of greater than about 200 Pa or greater than about 250 Pa, or greater than about 275 Pa, or from about 200 to about 310 Pa or from about 250 to about 310 Pa, or from about 275 to about 310 Pa, or from about 280 to about 300 or from about 280 to about 295, or from about 285 to about 290 Pa.

In a twenty-third aspect, the technology disclosed in this specification pertains to a sheared plant fiber or citrus fiber made by a process as described in any of the foregoing aspects.

In a twenty-fourth aspect, the technology disclosed in this specification pertains to the sheared plant fiber or citrus fiber of the twenty-third aspect being capable of forming an aqueous dispersion at 1% citrus fiber (d.b.) having a characteristic selected from the group consisting of:
(a) median particle size of less than about 45 microns, or less than about 43 microns, or from about 35 to about 45 microns, or from about 35 to about 43 microns, or from about 38 to about 43 microns;
(b) a 90th percentile particle size of from less than about 100 microns, or less than about 98 microns, or from about 80 to about 100 microns, or from about 85 to about 100 microns, or from about 90 to about 100 microns, or from about 95 to about 100 microns, or from about 95 to about 98 microns;
(c) a viscosity at 10 s⁻¹ of greater than about 3 Pa.s, or greater than about 3.1 Pa.s, or from about 3.0 to about 3.5, or from about 3.1 to about 3.2 Pa.s;
(d) a Bostwick distance of less than about 6.0 cm, or less than about 5.5, from about 4.5 to about 6.0, or from about 5.0 to about 6.0, or from about 5.0 to about 5.5;
(e) an elastic modulus (G′) at 1 rad/s of greater than about 200 Pa or greater than about 250 Pa, or greater than about 275 Pa, or from about 200 to about 310 Pa or from about 250 to about 310 Pa, or from about 275 to about 310 Pa, or from about 280 to about 300 or from about 280 to about 295, or from about 285 to about 290 Pa;
(f) a water holding capacity of from about 60 to about 100 g/g, or from about 70 to about 100 or from about 80 to about 100, or from about 85 to about 95, and
(g) mixtures thereof.

In a twenty-fifth aspect, the technology disclosed in this specification pertains to a use of a plant fiber or a citrus fiber as described in any of the foregoing aspects to provide viscosity to liquid, or to bind water, or to stabilize a suspension of solids in a liquid phase.

In a twenty-sixth aspect, the technology disclosed in this specification pertains to a composition comprising a plant fiber or citrus fiber as described in any foregoing aspect being an edible composition, cosmetic composition, a household composition, or an industrial composition.

In a twenty-seventh aspect, the technology disclosed in this specification pertains to the composition of the twenty-sixth aspect wherein the composition comprises a plant fiber or a citrus fiber as described in any of the foregoing claims and a second edible ingredient, wherein optionally the composition is selected from the group consisting of beverages, sauces, sauces, dressings, soft-baked and cold-pressed bars, and beverages and instant mixes, processed and packaged meat products and meat analog products, including plant based meat analog products, ice creams, frozen desserts, yogurts, baked goods, and gluten free goods.

The technology disclosed can be better understood by reference to the following definitions.

Reference in this specification to “Bostwick Distance” means the distance a sample flows under its own weight when filled in the trough of a Bostwick Consistometer and the gate opened. Bostwick distance may also be referred to as Bostwick viscosity within the art. Bostwick distance is commonly used in the art to quickly assess the consistency of a food product. A lower number means a sample is less flowable. Bostwick Distances are reported as the distance a substance flows, in centimeters, over 30 seconds.

Reference within this specification to “G′” means storage modulus. The storage modulus is a measure of elastic (solid-like) behavior of a viscoelastic material under oscillatory stress. It indicates how much energy is stored within a material via deformation of internal structures rather than dissipation through friction. A higher G′ typically indicates that a material has a more solid-like material character. Within this specification storage modulus is measured by a shearing stress applied at 1 rad/s. Within the art this amount of stress is understood to represent a “moderate” energy input - the material is not fully at rest but is also not sheared significantly

Reference in this specification to “depectinated” plant material means material obtained from a plant material having significant pectin in its native state, but has been subject to a process to remove at least a portion of the native state pectin.

Reference in this specification to “dewatering” means separation of a solid phase of a slurry from a liquid phase of a slurry.

Reference in this specification to a “dewatering efficiency” means the percent removal of liquid from a plant fiber material, or a citrus fiber material during dewatering. Within this specification dewatering efficiency is a percent measurement of the ratio of weight of the liquid removed during dewatering of a dispersed fiber versus the weight of the fiber dispersed in liquid. The liquid removed may be water or a mixture of water and other liquids. Within this specification an equation useful for calculating the dewatering efficiency is - dewatering efficiency (%) = (juice recovered (g)/ (juice recovered + material 100.

Use of “about” to modify a number in this specification is meant to include the number recited plus or minus 10%. Where legally permissible recitation of a value in a claim means about the value. Use of about in a claim or in the specification is not intended to limit the full scope of covered equivalents.

Recitation of the indefinite article “a” or the definite article “the” in this specification is meant to mean one or more unless the context clearly dictates otherwise.

While certain embodiments have been illustrated and described, a person with ordinary skill in the art, after reading the foregoing specification, can effect changes, substitutions of equivalents and other types of alterations to the methods, and of the present technology. Each aspect and embodiment described above can also have included or incorporated therewith such variations or aspects as disclosed regarding any or all the other aspects and embodiments.

The present technology is also not to be limited in terms of the aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to methods, conjugates, reagents, compounds, compositions, labeled compounds or biological systems, which can, of course, vary. All methods described herein can be performed in any suitable order unless otherwise indicated herein or otherwise clearly contradicted by context. It is also to be understood that the terminology used herein is for the purpose of describing aspects only and is not intended to be limiting. Thus, it is intended that the specification be considered as exemplary only with the breadth, scope and spirit of the present technology indicated only by the appended claims, definitions therein and any equivalents thereof. No language in the specification should be construed as indicating any non-claimed element as essential.

The embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the technology. This includes the generic description of the technology with a proviso or negative limitation removing any subject matter from the genus, regardless of whether the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member, and each separate value is incorporated into the specification as if it were individually recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The technology disclosed in this specification is further described with reference to examples, which are intended to be illustrative, and are not intended to limit the full scope of the claims and their equivalents.

The following three examples evaluate (1) the effect of shear on the ease of recovery of citrus fiber from water, the (2) effect of shear on citrus fiber recovered from isopropyl alcohol, and the (3) effect on dewatering efficiency from use of various amounts of isopropyl alcohol.

### EXAMPLE 1 - EFFECT OF SHEAR ON RECOVER OF CITRUS FIBER FROM WATER

Example 1 evaluates the effect of shear on the ability to recover citrus fiber by preparing Sample 1, which is depectinated lime peel that was sheared in water.

110 g of dried lime peel were dispersed in 2 L of deionized water. The dispersion was heated to 70° C. and pH adjusted to 1.8 to partially hydrolyze and solubilize pectin. After 4 hours, the dispersion was filtered using a wine press and muslin bag to separate pectin juice from insoluble peel components to produce a spent peel material. The spent peel filter material was tested by thermal balance and found to contain 89% moisture and 11% solids.

After diluting 510 g of spent peel material to 1250 g with water and treating with 20 mL 15% peracetic acid at 70° C. for 1 hour, the whitened slurry was processed using a Silverson LM5-A shear mixer at 10,000 rpm for 5 minutes using the 2 mm circular shearing screen. The sheared aqueous mixture was transferred to a muslin bag to dewater. While pressing with a wine press, the finer homogenized fibers passed through the pores of the muslin bag. The slurry was transferred to a 50-micron polyester filter cloth and again pressed within the wine press. No fluid could be extracted despite applying the maximum hand pressure.

The result of this example illustrates the difficulty in recovering sheared citrus fiber from water.

### EXAMPLE 2 - EFFECT OF SHEAR ON ISOPROPYL ALCOHOL RECOVERED CITRUS FIBERS

Example 2 evaluates the effect of shear on citrus fiber dispersed in alcohol solution by comparing an experimental sample (sample 2) that is sheared in isopropyl alcohol against an internally made control sample (sample 3) that is unsheared but was washed in isopropyl alcohol and against an unsheared commercial control (sample 4).

### Sample Preparation

### Sample 2 - Depectinated Lime Peel Sheared in Isopropyl Alcohol

110 g of dried lime peel were dispersed in 2 L of deionized water. The dispersion was heated to 70° C. and pH adjusted to 1.8 to partially hydrolyze and solubilize pectin. After 4 hours, the dispersion was filtered using a wine press and muslin bag to separate pectin juice from insoluble peel components to produce a spent peel material. The spent peel filter material was tested by thermal balance and found to contain 89% moisture and 11% solids.

510 g of this spent peel material was transferred to an IKA LR1000 reactor and brought to 1250 g with deionized water. The mixture was heated to 70° C. while gently stirring at 30 rpm. 20 mL of 15% peracetic acid was added to whiten the spent peel slurry. After 1 hr the whitened peel slurry was dewatered using a wine press and muslin cloth to create a filter material. The spent peel filter material was tested by thermal balance and found to contain 90% moisture and 10% solids. 510 g of filter material was returned to the reactor and dispersed with a volume of 510 mL of 98% isopropyl alcohol. The alcoholic dispersion was processed with a Silverson LM5-A high shear mixer at 10,000 rpm to break up citrus fiber peel sections. The sheared slurry was then passed through a colander with 2 mm hole size to remove hard pieces of seeds which potentially may clog homogenizing equipment. The screened slurry was then introduced to an APV1000 high pressure homogenizer and passed one time across the homogenizing nozzle at a pressure of 14,000 psi. An aliquot of this homogenized slurry was filtered using a wine press and 50-micron polyester filter cloth to produce 200 g of fiber material retentate and 410 g of alcohol/water filtrate. Alcohol consumption to this first filtering stage was 1 mL isopropyl alcohol per 1 g of spent peel material. The filter material was dispersed a 2^{nd} time in an additional 200 g of isopropyl alcohol. The slurry was stirred at room temperature for 30 minutes and then filter a second time to produce a twice alcohol washed fiber material. The material was broken up and dried within the LR1000 with the bottom plate set to 50° C., constant stirring at 30 rpm and with dry compressed air blowing through the reactor for 1 hour.

### Sample 3 - Alcohol Washed, Unsheared Depectinated Lime Peel

Spent lime peel material was prepared as in Example1 and 2. After similarly diluting 510 g of spent peel material to 1250 g with water and treating with 20 mL 15% peracetic acid at 70° C. for 1 hour, the whitened peel slurry was dewatered using a wine press and muslin cloth to create a filter material. The spent peel filter material was tested by thermal balance and found to contain 91% moisture and 9% solids. 515 g of filter material was returned to the reactor and dispersed with a volume of 515 mL of 98% isopropyl alcohol. This alcoholic slurry was filtered using a wine press and 50-micron polyester filter cloth to produce a non-homogenized filter material. The filter material was dispersed a 2^{nd} time in an equal proportion isopropyl alcohol to example 1. The slurry was stirred at room temperature for 30 minutes and then filter a second time to produce a twice alcohol washed non-homogenized fiber material. The material was broken up and dried within the LR1000 with the bottom plate set to 50° C., constant stirring at 30 rpm and with dry compressed air blowing through the reactor for 1 hour.

### Sample 4 - Commercial Control

Sample 4 is an unsheared commercially available control citrus fiber.

### Results - Functional Properties of Samples 2, 3, and 4

The fibers of Samples 2, 3, and 4 were added at 1% dry basis in deionized water with 300 ppm potassium sorbate as a preservative. The preparations were dispersed using a Silverson LM5-A laboratory mixer at 10,000 rpm for 10 minutes. To determine Bostwick viscosity, a Bostwick consistometer was leveled and the trough filled with the wet fiber dispersions. The trough gate was opened and the distance the fiber dispersion traveled over 30 seconds was recorded, with smaller distances indicating greater viscosity. The rheological properties of the fiber dispersions were also assessed with a TA Instruments AR-G2 rheometer using a vane geometry. A dynamic amplitude sweep was performed to identify the linear viscoelastic range. A dynamic frequency sweep was then performed at an amplitude selected from the linear viscoelastic range, and the G′ value at 1 rad/s was obtained. The viscosity value at a shear rate of 10 s-1 was extracted. The wet particle size distribution of the fibers dispersions was determined using a Malvern laser diffractometer, and values for the median and 90% percentile particle size were obtained. Results of these measurements are reported in Table 1.

TABLE 1

Functional Characteristics of Sheared and Unsheared Fibers

| Sample | Bostwick Distance (cm) | G′ at 1 rad/s (Pa) | Viscosity at 10 s-1 (Pa.s) | Median Particle Size (microns) | 90% Percentile Particle Size (microns) |
|---|---|---|---|---|---|
| Sample 2 (alcohol sheared citrus fiber) | 5.2 | 287.0 | 3.15 | 41.9 | 96.9 |
| Sample 3 (unhomogenized alcohol recovered citrus fiber) | 6.2 | 141.1 | 2.78 | 53.2 | 138.2 |
| Example 4 (commercial control) | 7.4 | 115.4 | 2.16 | 70.2 | 146.4 |

As seen the material sheared in water and isopropanol produced thicker mixtures when re-slurried and have smaller particle size than unsheared samples. The results of this example demonstrate that citrus fiber sheared in water/isopropanol mixture can be readily functionalized compared to unsheared plant material, and while being highly recoverable from the shearing process slurry.

### EXAMPLE 3 - DEWATERING EFFICIENCY OF FIBER HOMOGENIZED IN ISOPROPYL ALCOHOL

This example illustrates how dewatering efficiency change with concentration of isopropyl alcohol.

Sample 5 is a control sample prepared like Sample 1 and further illustrates the difficulty of dewatering a citrus peel sheared in water.

Samples 6, 7 and 8 were prepared like Sample 2, but with various concentrations of isopropyl alcohol but filtered only once through the 50-micron polyester filter cloth. Sample 6 is a 50% (v/v) mixture of alcohol and water, Sample 7 is a 75% water mixture (v/v), and Sample 8 uses an 87.%% water mixture (v/v). Dewatering efficiency is reported in Table 2 and is the percent juice recovered (g) from pressed sheared material versus total weight of sheared material and juice (i.e. dewatering efficienty (%) = (juice (g)/ juice (g) +material (g))*100).

TABLE 2

Dewatering Efficiency

| fiber amount (g) | total volume (ml) | water fraction (% v/v) | Juice (g) | Material (g) | Dewatering efficiency (%) |
|---|---|---|---|---|---|
| 35.23 | 1175 | 100 | 30.64 | 650 | 4.5 |
| 35.23 | 1175 | 50 | 322 | 499 | 39.2 |
| 35.23 | 1175 | 75 | 204.4 | 679 | 23.1 |
| 35.23 | 1175 | 87.5 | 205 | 660 | 23.7 |

As seen, while dewatering efficiency increase with increased isopropyl alcohol concentration significant improvements in dewatering efficiency are observed using limited isopropyl alcohol.

What is claimed is:

1. A method for increasing the dewatering efficiency of a sheared plant fiber comprising:
(i) forming a slurry having a solid phase and a liquid phase comprising a comminuted depectinated plant material an organic solvent and an aqueous phase;
(ii) applying shear to the slurry; and
(iii) recovering the plant fiber from the slurry.

2. The method of claim 1 wherein the solvent is an alcohol.

3. The method of claim 1 wherein the depectinated plant fiber has a pectin content of less than about 19% by weight (d.b.).

4. The method of claim 1 wherein the slurry’s liquid phase comprises water and organic solvent, optionally wherein the slurry comprises from from about 5% to about 25%.

5. The method of claim 1 wherein the shear energy is applied via a pressure drop across a restricted orifice or nozzle and the pressure drop is at least about 100 to about 5000 bar.

6. The method of claim 1 wherein the shear energy is applied to the slurry by a rotor/stator homogenizer rotating at from about 1,000 to about 20,000 rpm.

7. The method of claim 1 wherein the slurry is sheared in one pass, at least one pass, or at least 2 passes.

8. The method of claim 1 wherein the slurry’s solid phase comprises about 0.25 to about 15% (d.b.).

9. The method of claim 1 wherein recovering the plant fiber removes at least about 10% of the liquid from the dispersed fiber.

10. The method of claim 1 further comprising drying the recovered plant fiber, wherein, optionally, the dried plant fiber has moisture content of less than about 10%.

11. The method of claim 1 further comprising washing the depectinated plant material prior to applying the shear plant material, optionally wherein the washing step whitens the plant material.

12. The method of claim 1 further comprising washing the depectinated plant material in a solution comprising from about 5% to about 30% v/v of an organic acid, wherein the washing is performed prior to applying the shear.

13. A sheared plant fiber or citrus fiber made by a process as described in claim 1 capable of forming an aqueous dispersion at 1% fiber (d.b.) that has one or more characteristics selected from the group consisting of:
(a) median particle size of less than about 45 microns;
(b) a 90th percentile particle size of from less than about 100 microns;
(c) a viscosity at 10 s⁻¹ of greater than about 3 Pa.s
(e) an elastic modulus (G′) at 1 rad/s of greater than about 200 Pa and
(f) a water holding capacity of from about 60 to about 100 g/g.

14. A sheared plant fiber or citrus fiber capable of forming an aqueous dispersion at 1% fiber (d.b.) having a median particle size of less than about 45 microns.

15. The sheared plant fiber or citrus fiber of claim 14 capable of forming an aqueous dispersion at 1% fiber content (d.b.) having a 90th percentile particle size of from less than about 100 microns.

16. The sheared plant fiber or citrus fiber of claim 14 capable of forming an aqueous dispersion at 1% fiber (d.b.) having a viscosity at 10 s⁻¹ of greater than about 3 Pa.s.

17. The sheared plant fiber or citrus fiber of claim 14 capable of forming an aqueous dispersion at 1% fiber (d.b.) having a Bostwick distance of less than about 6.0 cm.

18. The sheared plant fiber or citrus fiber of claim 14 capable of forming an aqueous dispersion at 1% fiber (d.b.) having an elastic modulus (G′) at 1 rad/s of greater than about 200 Pa.

19. The sheared plant fiber or citrus fiber of claim 14 capable of forming an aqueous dispersion at 1% fiber (d.b.) having a water holding capacity of from about 60 to about 100 g/g.
